(19) United States
(12) Patent Application Publication
(10) Pub. No.: US 2007/0086987 A1
(43) Pub. Date: Apr. 19, 2007

(54) Skeletal musle-derived cells and methods related thereto

(76) Inventors: Peter C. Yaeger, Natick, MA (US); Jeffrey D. Stewart, Milford, MA (US); Bruce M. Wentworth, Northborough, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/579,508

(22) PCT Filed: Nov. 15, 2004
(86) PCT No.: PCT/US04/38059

§ 371 Date: May 16, 2006

Related U.S. Application Data
(60) Provisional application No. 60/520,762, filed on Nov. 17, 2003.

(51) Int. Cl. A61K 35/34; C12N 5/06; C12N 5/08

(52) U.S. Cl. 424/93.7; 435/325; 435/366

(57) ABSTRACT

The disclosure provides methods of propagating skeletal muscle cell (SkMC) cultures enriched in differentiation-competent myoblasts that express normal levels of CD56 and reduced levels of desmin. The methods comprise culturing SkMCs in a mitogen-rich cell culture medium supplemented with TGF-β. The disclosure also provides therapeutic methods utilizing SkMCs propagated in TGF-β, e.g., methods of treating myocardial infarction by transplantation of autologous or allogeneic SkMCs.

This application claims priority to U.S. application Ser. No. 60/502,762, filed on Nov. 17, 2003, herein incorporated by reference in its entirety.

### FIELD OF THE INVENTION

The present invention relates to methods of propagating skeletal muscle-derived cells, and in particular, cells intended for implantation into injured heart tissue. The invention further relates to cell culture medium compositions that contain TGF-β.

### BACKGROUND OF THE INVENTION

Heart failure, mostly due to myocardial insufficiency, is a frequent and life-threatening condition, despite medical and surgical advances. Therapeutic application of autologous human skeletal muscle cells (HuSkMCs) to mitigate the deterioration of cardiac function resulting from myocardial infarction has shown promise in several preclinical and clinical studies (see, e.g., Atkins et al. (1999) Heart Lung Transplant., 18:1173-1180; Hutcheson et al. (2000) Cell Transplant., 9:359-368; Pouzet et al. (2001) Circulation, 102:210-215; Scorsin et al. (2000) J. Thorac. Cardiovasc., 119:1169-1175; Jain et al. (2001) Circulation, 103:1920-1927; Ghostine et al. (2002) Circulation 106 (Suppl.):I-131-I-136; Thompson et al. (2003) Circulation, 108 (Suppl.):II-264-II-271; Menasche et al. (2001) Lancet, 357:279-280; Menasche (2003) Cardiovasc. Res., 58:351-357; Menasche et al. (2003), J. Am. Coll. Cardiol., 41:1078-1083; Hagege et al. (2003) Lancet, 361:491-492; Pagani et al. (2003) J. Am. Coll. Cardiol., 41:879-888). In these studies, skeletal muscle cells (SkMCs), obtained from skeletal muscle biopsies, are propagated in vitro and subsequently injected into damaged heart tissue. A correlation between the higher number of SkMCs injected (from 7×10⁵ to 7×10⁶ cells) and improved cardiac function has been established in a rat infarct model (Pouzet et al. (2001) Circulation, 104:I223-I228). Based on the relative weights of rat and human hearts, as many as 10⁹ HuSkMCs may be required for therapeutic efficacy in human patients. To this end, HuSkMCs may need to be propagated for several passages, since the number of cells available from biopsies is generally limited. The challenge is not only to consistently produce a large number of cells but also to reliably characterize the identity and differentiation state of cells in culture.

Skeletal muscle contains satellite cells, which are quiescent myoblast precursors that reside between the basal lamina and sarcolemma of mature myofibers (Allen et al. (1997) Meth. Cell Biol., 52:155-176). In growing or damaged muscle, satellite cells are activated to become proliferating myoblasts, which ultimately undergo differentiation into mature muscle fibers (Campion (1984) Int. Rev. Cytol., 87:225-251). In cell culture, activation of satellite cells and their propagation as myoblasts may be achieved by enzymatic dissociation of cells in skeletal muscle and cultivation in mitogen-rich culture medium (Allen et al., supra).

Cells of non-myoblast lineage, primarily fibroblasts, are also released from muscle tissue upon enzymatic dissociation. Fibroblasts co-propagate with myoblasts and can potentially dominate the cultures. Differentiation of myoblasts into mature myocytes is accompanied by the cessation of their proliferation (Nadal-Ginard et al. (1978) Cell, 15:855-864), which, in turn, enables overgrowth of fibroblasts in serially propagated HuSkMC cultures. Because data suggest that it is the myoblasts of skeletal muscle-derived cultures that contribute to cardiac contractility after implantation into injured heart tissue (see, e.g., Pouzet et al. (2001) Circulation, 102:210-215), one goal in HuSkMC propagation is to minimize the presence of fibroblasts.

Myoblast differentiation is typically induced by reduction of serum and other mitogens in the culture medium (Allen et al., supra) but some spontaneous differentiation occurs even in mitogen-rich cultures, especially at high cell density. Therefore, another objective in HuSkMCs propagation is to suppress differentiation of myoblasts while maintaining them in a proliferative state.

Transforming growth factor beta (TGF-β), a growth factor found in normal and transformed tissues, is reported to suppress or induce myoblast differentiation depending on the biological system under study. For example, TGF-β has been reported to suppress myoblast differentiation in a number of systems, mainly in studies performed on established clonal cell lines or embryo-derived myoblasts (Florini et al. (1986) J. Biol. Chem., 261:16509-16513; Massague et al. (1986) Proc. Natl. Acad. Sci. USA, 83:8206-8210; Rousse et al. (2001) J. Biol. Chem., 276:46961-46967; Liu et al. (2001) Genes Dev., 15:2950-2966; and Olson et al. (1986) J. Biol. Chem., 103:1799-1805). Contrary to these findings, other investigators have reported that TGF-β stimulates myoblast differentiation under low cell density conditions (De Angelis et al. (1998) Proc. Natl. Acad. Sci. USA, 95:12358-12363), in serum-free media (Schofield et al. (1990) Exp. Cell. Res., 191:144-148), and in mitogen-rich medium used to culture the L₆E₉ myoblast cell line (Zentella et al. (1992) Proc. Natl. Acad. Sci. USA, 89:5176-5180).

The three mammalian isoforms of TGF-β (TGF-β1, -β2, and -β3) generally have similar effects on cells in vitro, but appear to have distinct biological roles in vivo (McLennan et al. (2002) Int. J. Dev. Biol., 46:559-567). The temporal and spatial distribution of the TGF-β isoforms in developing and regenerating muscle, along with other evidence, implicates TGF-β in myoblast differentiation by mediating myoblast fusion in vivo (McLennan et al., supra).

Therefore, there exists a need in the art to gain more understanding of the role of TGF-β in myoblast differentiation and to develop clinically suitable methods for propagating HuSkMCs.

### SUMMARY OF THE INVENTION

The present invention provides methods for reversibly suppressing myoblast differentiation into myocytes during propagation of skeletal muscle cell (SkMC) cultures, while maintaining myoblast proliferation.

The invention further provides methods for determining the constituent cell identity and/or differentiation state of cells in a SkMC culture.

The invention yet further provides methods for enriching SkMC cultures in differentiation-competent myoblasts expressing reduced levels of myocyte differentiation markers. The invention provides such enriched SkMC cultures and therapeutic methods utilizing these cultures.

Additional aspects and advantages of the invention will be set forth in part in the following description, and in part will be understood from the description or may be learned by practice of the invention.

### BRIEF DESCRIPTION OF THE FIGURES

FIG. 1 depicts results of dual-fluorescent immunolabeling for desmin and CD56 performed on 3rd passage HuSkMCs of Strain A. Flow cytometric analysis reveals two major populations, one expressing both myoblast markers (Des+ and CD56+) and one expressing neither marker (Des− and CD56−).

FIG. 2 illustrates the effect of TGF-β2 on myoblast markers as a function of time in TGF-β2. HuSkMCs of strain A were propagated for 0, 0.17, 1, 2, or 5 days in 2nd passage, then detached and subjected-to fluorescent immunolabeling for detection of the myoblast markers desmin and CD56. Mean fluorescence of the desmin-positive (solid line) and CD56-positive (dashed line) myoblast populations. Results were averaged from duplicate cultures. Error bars identify the range of values. While the percentage of Des+ and CD56+ cells and the level of CD56 expression were substantially unaffected by TGF-β, desmin expression gradually declined.

FIG. 3 illustrates the effect of TGF-β2 on creatine kinase activity. A sample of cells from the same Strain A cultures was lysed at the same time they were harvested for flow cytometry analysis (FIG. 2), then analyzed for creatine kinase activity. These cells had been propagated 5 days with 1 ng/ml TGF-β2 present during the final 0, 0.17, 1, 2, or 5 days of culture, as indicated. Results were averaged from duplicate cultures. Error bars identify the range of values. Note the similarity in decay of creatine kinase activity (FIG. 3) and desmin expression (FIG. 2).

### DETAILED DESCRIPTION OF THE INVENTION

In order that the present invention may be more readily understood, certain terms are first defined. Additional definitions are set forth throughout the detailed description.

The term “CD56-positive,” when used to describe cells, refers to cells expressing detectable levels of CD56. Likewise, the term “desmin-positive” refers to cells expressing detectable levels of desmin. Expression can be detected at the protein or RNA levels using methods known in the art and/or as described in the Examples.

The term “mitogen-rich medium” refers to a medium comprising at least 5% serum or combinations of various sera.

The term “TGF-β,” unless otherwise specifically indicated, refers to any one or more isoforms of TGF-β. Currently, there are 5 known isoforms of TGF-β (TGF-β1-β5), all of which are substantially homologous among each other (60-80% identity), form homodimers, and act upon common TGF-β receptors (TβR-I, TβR-II, TβR-IIB, and TβR-III). TGF-β is highly conserved among species. For example, porcine, simian, and human mature TGF-β1's (112 amino acids) are identical, and mouse and rat TGF-β1 differ only by one amino acid from human. The structural and functional aspects of TGF-β are well known in the art (see, for example, Oppenheim et al. (eds) Cytokine Reference, Academic Press, San Diego, Calif, 2001, pp. 719-746). Only TGF-β1, TGF-β2, and TGF-β3 are found in mammals. A partial listing of protein accession number for the three isoforms is provided in Table 1.

TABLE 1

Accession numbers

| | TGF-β1 | TGF-β2 | TGF-β3 |
|---|---|---|---|
| Human | PO1137 | P08112 | P109600 |
| Mouse | P04202 | P27090 | P171125 |
| Rat | AAD20222 | AAD24484 | Q07258 |
| Porcine | AAA616 | AAB03850 | P15203 |
| Simian | P09533 | WFMKB2 | |

Unless otherwise indicated, the amounts of TGF-β stated refer to the amounts of active TGF-β added to the medium and do not include TGF-β naturally present in the serum, the amount of which may vary depending on the serum source. The reported serum concentrations of TGF-β1, the most prevalent form of TGF-β, vary between 1 and 33 ng/ml (Kyrtsonis et al. (1998) Med. Oncol., 15:124-128). According to the manufacturer, the amount of TGF-β1 in the Defined Fetal Bovine Serum utilized in the Examples is, on average, 21 ng/ml (Wight (2000) Art to Science, Vol. 19(3):1-3). However, most TGF-β naturally present in various sera is in the inactive form, i.e., with the propeptide non-covalently bound to the mature form of the growth factor.

Since TGF-β exhibits diverse bioactivities, various assays can be used to detect and quantitate TGF-β amount and/or activity. Examples of some of the more frequently used in vitro bioassays for TGF-β, activity include:
(1) induction of colony formation of NRK cells in soft agar in the presence of EGF (Roberts et al. (1981) Proc. Natl. Acad. Sci. USA, 78:5339-5343);
(2) induction of differentiation of primitive mesenchymal cells to express a cartilaginous phenotype (Seyedin et al. (1985) Proc. Natl. Acad. Sci. USA, 82:2267-2271);
(3) inhibition of growth of Mv1Lu mink lung epithelial cells (Danielpour et al. (1989) J. Cell. Physiol., 138:79-86) and BBC-1 monkey kidney cells (Holley et al. (1980) Proc. Natl. Acad. Sci. USA 77:5989-5992);
(4) inhibition of mitogenesis of C3H/HeJ mouse thymocytes (Wrann et al. (1987) EMBO J., 6:1633-1636);
(5) inhibition of differentiation of rat L6 myoblast cells (Florini et al. (1986) J. Biol. Chem., 261:16509-16513);
(6) measurement of fibronectin production (Wrana et al. (1992) Cell, 71:1003-1014);
(7) induction of plasminogen activator inhibitor I (PAI-1) promoter fused to a luciferase reporter gene (Abe et al. (1994) Anal. Biochem., 216:276-284); and
(8) sandwich enzyme-linked immunosorbent assays (Danielpour et al. (1989) Growth Factors, 2:61-71).

The terms “primary culture” and “primary cells” refer to cells derived from intact or dissociated tissues or organ fragments. A culture is considered primary until it is passaged (or subcultured) after which it is termed a “cell line” or a “cell strain.” The term “cell line” does not imply homogeneity or the degree to which a culture has been characterized. A cell line is termed “clonal cell line” or “clone” if it is derived from a single cell in a population of cultured cells. Unless otherwise indicated, the terms “skeletal muscle cells (SkMCs)” and “SkMC culture” refer to both primary and passaged skeletal muscle cells. The terms “SkMCs” and “SkMC culture” refer to cells isolated from skeletal muscle as well as non-clonal cells purified, separated, and/or subcultured therefrom, including (but not limited to) purified myoblasts. The term “high density” refers to cell density of more than 50,000 cells/cm² or 50% confluence.

The term “passage” and its cognates refer to a process of transferring cells to a new culture vessel so as to propagate the cell population or set up replicate cultures. Depending on the context, the term “passage” may also refer to cells in culture that have been passaged, and/or to the time span between sequential passages. Unless indicated otherwise, “1st passage” refers to primary culture; “2nd passage” refers to cells passaged from a primary culture; “3rd passage” refers to cells passaged from a 2nd passage culture, and so on.

The invention is based, in part, on the discovery and demonstration that TGF-β2 reversibly suppresses myoblast differentiation in serially propagated cultures of adult HuSkMCs, even in high density cultures. Suppression of myoblast differentiation was confirmed by a reduction in expression of creatine kinase, an established marker of myoblast differentiation. These results indicate that TGF-β may be used to suppress myoblast differentiation during large-scale production of HuSkMCs for clinical use. By inhibiting myoblast differentiation during serial propagation of SkMC, TGF-β maintains the myoblast population in a proliferative, differentiation-competent state. The ability of TGF-β to suppress myoblast differentiation even after culture of SkMCs to high density allows for less frequent passaging and/or smaller tissue culture surface areas during the serial propagation of SkMCs. Propagation of SkMCs in TGF-β may also facilitate engraftment of myoblasts once injected into injured heart tissue, since undifferentiated cells are thought to exhibit enhanced proliferation and motility during the initial stages of engraftment.

Accordingly, one aspect of the invention is a method of propagating SkMCs in culture. In certain embodiments, the SkMCs are primary or passaged cells obtained from an adult mammal, for example, HuSkMCs. A related aspect of the invention is a method for enriching SkMC cultures in differentiation-competent myoblasts expressing reduced levels of myocyte differentiation markers. The methods comprise culturing SkMCs in a mitogen-rich cell culture medium supplemented with an amount of TGF-β effective to reversibly suppress myoblast differentiation. In various embodiments, the SkMCs are primary or passaged cells, cultured in a medium supplemented with TGF-β, for example, for at least 12, 24, 36, 48, 72, 96, 120, 144, 168 hours or longer in 1st, 2nd, 3rd, 4th, 5th, 6th, 7th and/or subsequent passages. In further embodiments, in one or more passages, prior to passaging and/or harvest, cells are grown to a density of over 30, 35, 40, 45, 50, 55, 60, 65, 70, 75, 80, 85, 90, 95% or higher confluence as measured by the percentage of culture surface occupied by cells, or over 0.1, 0.25, 0.5, 0.75, 1, 1.25, 1.5, 1.75, 2, 2.1, 2.3, 2.5, 2.75, 3, 3.25, 3.5, 3.75, 4, 5 or greater ×10⁵ cells/cm². In illustrative embodiments, cells are grown for 1, 2, or 5 days in 2nd passage in the presence of TGF-β.

In various embodiments, TGF-β is one of, or any combination of, TGF-β1, TGF-β2, and TGF-β3, or heterodimers thereof. TGF-β4 and TGF-β5 may also be used. The amount of TGF-β with which culture media is supplemented is effective to suppress myoblast differentiation. In some embodiments, the effective amount is 0.01, 0.05, 0.1, 0.5, 1, 1.5, 2, 3, 4, 5, 10, 20, or 40 ng/ml or is chosen from the ranges of 0.01 to 200, 0.01 to 100, 0.01 to 50, 0.01 to 20, 0.2 to 50, 0.2 to 20, 0.2 to 10, 0.2 to 5, 0.2 to 2, 0.5 to 5, and 0.5 to 2 ng/ml. In an illustrative embodiment, the medium is supplemented with 1 ng/ml TGF-β2 .

The invention is further based, in part, on the discovery and demonstration that the reduction in desmin expression by CD56-positive myoblasts correlates with the suppression of myoblast differentiation by TGF-β, whereas expression of CD56 is unaffected by TGF-β.

Clonal growth and differentiation of skeletal muscle cells in culture was first reported by Konigsberg (1963) Science, 140:1273. During differentiation, myoblasts enter the post-mitotic G₀ phase and myoblast fusion (fusion-burst) becomes evident within 48 hours after plating. Around the time of fusion-burst, transcription of muscle-specific genes (e.g., creatine kinase) is upregulated (Paterson et al. (1972) cell, 17:771; Delvin et al. (1978) Nature, 270:725). Creatine kinase activity, which provides energy for muscle contraction via ATP regeneration, is a long-established quantifiable marker of myoblast differentiation and correlates with myoblast fusion (Shainberg et al. (1971) Dev. Biol., 25:1-29).

The intermediate filament protein desmin is expressed in proliferating skeletal myoblasts (Kaufman et al. (1988) Proc. Natl. Acad. Sci. USA, 85:9606-9610; Lawson-Smith et al. (1998) J. Anat., 192:161-171) and is prevalent in mature myocytes of skeletal muscle (Lazarides et al. (1976) Proc. Natl. Acad. Sci. USA, 73:4344-4348). Upregulation of desmin is a signal of myoblast differentiation. In contrast, CD56 (also named NCAM or Antigen Leu-19) is expressed constitutively in proliferating myoblasts (IIIa et al. (1992) Ann. Neurol., 31:46-52; and Belles-Isles et al. (1993) Eur. J. Histochem., 37:375-380), but is absent in mature muscle (Schubert et al. (1989) Proc. Nati. Acad. Sci. USA, 86:307-311). Other cells also express CD56, including certain lymphocytes and neurons, but not fibroblasts. Desmin and CD56 are both considered reliable markers for myoblasts among cells cultured from skeletal muscle.

The invention is based, in part, on the discovery and demonstration that two populations account for nearly all cells within skeletal muscle cultures: (1) CD56⁺, desmin⁺, TE7⁻ cells; and (2) CD56⁻, desmin⁻, TE7⁺ cells. These two populations are myoblasts and fibroblasts, respectively. Desmin and CD56 are two markers of proliferating skeletal myoblasts. TE7 is a monoclonal antibody, which binds fibroblastic stromal cells of bone marrow (Cattoretti et al. (1993) Blood, 81:225-251) and thymic tissue sections (Haynes et al. (1984) J. Exp. Med., 159:1149-1168). The TE7 antigen is a marker of fibroblasts in vitro (Rosendal et al. (1994) J. Cell Sci., 102:29-37).

The invention is further based, in part, on the discovery and demonstration that the reduction in desmin expression by CD56-positive (CD56+) myoblasts correlates with the suppression of myoblast differentiation by TGF-β, whereas expression of CD56 is unaffected by TGF-β. Despite the loss of desmin, a generally accepted marker of myoblasts, TGF-β2 does not cause a loss of the myoblast phenotype via transdifferentiation into another cell type, as might have been expected (see, e.g., Katagiri et al. (1994) J. Cell Biol., 127:1755-1766).

Accordingly, another aspect of the invention is a method for evaluating the differentiation state of myoblasts in a SkMC culture. The method comprises determining the amount of desmin expressed by a population of CD56-positive cells in the SkMC culture, wherein the amount of desmin below a threshold level indicates the presence of undifferentiated myoblasts in the SkMC culture.

In a further aspect, the invention provides SkMCs propagated in a medium supplemented with TGF-β, according to the methods of the invention. SkMCs can be obtained from skeletal muscle of vertebrate species, including mammals (e.g., rat, murine, bovine, porcine, simian, and human) and non-mammals (e.g., avian). The term “adult” in reference to SkMCs, is used for SkMCs derived from a postnatal animal (e.g., the human) to distinguish these cells from embryonic SkMCs.

The compositions of the invention comprise cultured SkMCs enriched in differentiation-competent myoblasts that express normal levels of CD56 and reduced levels of desmin. In certain embodiments, desmin expression by CD56-positive myoblasts is reduced by at least 20, 30, 40, 50, 60, 70% or more, relative to (a) a control culture propagated without the supplementation with TGF-β and/or (b) the primary cells., In certain embodiments, desmin expression by CD56-positive myoblasts propagated in TGF-β is reduced by at least 20, 30, 40, 50, 60, 70% or more, relative to CD56-positive cells in the same culture prior to the addition of TGF-β.

The compositions of the invention further comprise cultured SkMCs that express reduced amounts of creatine kinase. In certain embodiments, creatine kinase expression by the SkMCs is reduced by at least 20, 30, 40, 50, 60, 70% or more, relative to a control culture propagated without the supplementation with TGF-β. In certain embodiments, creatine kinase expression by SkMCs propagated in TGF-β is reduced by at least 20, 30, 40, 50, 60, 70% or more, relative to the same SkMCs in culture prior to the addition of TGF-β. Expression levels are referenced per cell number of relevant cell population.

The levels of CD56, desmin and creatine kinase can be measured at the RNA or at the protein level. RNA levels may be determined by, for example, quantitative real time PCR (RT-PCR), Northern blotting, or another method for determining RNA levels, for example, as described in Sambrook et al. (eds.) Cloning: A Laboratory Manual, 2nd ed., Cold Spring Harbor Laboratory Press, 1989. CD56, desmin, and creatine kinase expression levels may be measured at the protein level using flow cytometry (fluorescence-activated cell sorting (FACS)), Western blotting, ELISA, immunohistochemistry, enzymatic activity assays (e.g., creatine kinase assay), or another method for determining protein levels, for example, as described in Current Protocols in Molecular Biology (Ausubel et al. (eds.) New York: John Wiley and Sons, 1998, or in the Examples.

Methods for cell isolation and culture, including methods for isolation and culture of SkMCs are known in the art and can be performed, for example, as described in Davis (ed.) Basic Cell Culture, 2nd ed., Oxford University Press Inc., New York, 2002, pp. 244-247, or in the Examples. Generally, cells are maintained in a culture medium providing essential nutrients, vitamins, co-factors necessary to support cellular functions. Optimal culture conditions for most mammalian cells typically include pH of 7.2-7.5, osmolarity of 280-320 nOsmol/kg, 2-5% CO₂, and temperature of 32-37° C. Typically, skeletal muscle cultures are propagated in mitogen-rich media that contain 5-20, 7-15, or 10% of the serum. Sera can be obtained from human, bovine, horse, sheep, goat, chicken, or other sources. Selection of serum and serum batches are based, in part, on empirical evaluation by the user. Batch-to-batch variability in cell yields within ±20% would normally be considered satisfactory.

A skilled artisan will also appreciate that the media used in the methods of the invention may be prepared from a variety of known media, e.g., Eagle's medium (Eagle (1955) Science, 122:501), Dulbecco's Minimum Essential medium (Dulbecco et al. (1959) Virology, 8:396), Ham's medium (Ham (1963) Exp. Cell Res., 29:515), L-15 medium (Leibvitz (1963) Amer. J. Hyg., 78:173), McCoy 5A medium (McCoy et al. (1959) Proc. Exp. Biol. Med., 100:115), RPMI medium (Moore et al. (1967) J. A. M. A., 199:519), Williams' medium (Williams (1971) Exp. Cell Res., 69:106-112), NCTC 135 medium (Evans et al. (1968) Exp. Cell Res., 36:439), Waymouth's medium MB752/1 (Waymouth (1959) Natl. Cancer Inst., 22:1003), etc. These media may be used singularly or as mixtures in suitable proportions to prepare cell culture media. Alternatively, media can be prepared from individual chemicals and/or from other media and growth supplements, as for example, specified in Table 2. The invention is not limited to media of any particular consistency and encompasses the use of media ranging from liquid to semi-solid compositions. The methods of this invention are suitable for cells growing in cultures under various conditions including (but not limited to) monolayers, multilayers, on solid support, in suspension, and in 3D cultures.

TABLE 2
Compositions of Basal Media

| | DMEM | RPMI-1640 | Ham's F-12 |
|---|---|---|---|
| | 1x Liquid, | 1x Liquid, | 1x Liquid, |
| | mg/L | mg/L | mg/L |
| Inorganic Salts | | | |
| CaCl₂ (anhyd.) | 200.00 | | 33.22 |
| Ca(NO₃)₂•4H₂O | | 100.00 | |
| CuSO₄•5H₂O | | | 0.0024 |
| Fe(NO₃)₂•9H₂O | 0.10 | | |
| FeSO₄•7H₂O | | | 0.83 |
| KCl | 400.00 | 400.00 | 223.60 |
| MgSO₄ (anhyd.) | 97.67 | 48.84 | |
| MgCl₂ (anhyd.) | | | 57.22 |
| NaCl | 6400.00 | 6000.00 | 7599.00 |
| NaHCO₃ | 3700.00 | 2000.00 | 1176.00 |
| NaH₂PO₄•H₂O | 125.00 | | |
| Na₂HPO₄ (anhyd.) | | 800.00 | 142.00 |
| ZnSO₄•7H₂O | | | 0.86 |
| Other Components | | | |
| D-Glucose | 4500.00 | 2000.00 | 1802.00 |
| Glutathione (reduced) | | 1.00 | |
| Hypoxanthine Na | | | 4.77 |
| Linoleic Acid | | | 0.084 |
| Lipoic Acid | | | 0.21 |
| Phenol Red | 15.00 | 5.00 | 1.20 |
| Putrescine 2HCl | | | 0.161 |
| Sodium Pyruvate | | | 110.00 |
| Thymidine | | | 0.70 |
| Amino Acids | | | |
| L-Alanine | | | 8.90 |
| L-Arginine | | 200.00 | |
| L-Arginine•HCl | 84.00 | | 211.00 |
| L-Asparagine•H₂O | | | 15.01 |
| L-Asparagine (free | | 50.00 | |
| base) | | | |
| L-Aspartic Acid | | 20.00 | 13.30 |
| L-Cystine•2HCl | 63.00 | 65.00 | |
| L-Cysteine•HCl•H₂O | | | 35.12 |
| L-Glutamic Acid | | 20.00 | 14.70 |
| L-Glutamine | 584.00 | 300.00 | 146.00 |
| Glycine | 30.00 | 10.00 | 7.50 |
| L-Histidine•HCl•H₂O | 42.00 | | 21.00 |
| L-Histidine (free base) | 1.00 | 5.00 | |
| L-Hydroxyproline | | 20.00 | |
| L-Isoleucine | 105.00 | 50.00 | 4.00 |
| L-Leucine | 105.00 | 50.00 | 13.10 |
| L-Lysine•HCl | 146.00 | 40.00 | 36.50 |
| L-Methionine | 30.00 | 15.00 | 4.50 |
| L-Phenylalanine | 66.00 | 15.00 | 5.00 |
| L-Proline | | 20.00 | 34.50 |
| L-Serine | 42.00 | 30.00 | 10.50 |
| L-Threonine | 95.00 | 20.00 | 11.90 |
| L-Tryptophan | 16.00 | 5.00 | 2.00 |
| L-Tyrosine•2Na₂H₂O | 104.00 | 29.00 | 7.81 |
| L-Valine | 94.00 | 20.00 | 11.70 |
| Vitamins | | | |
| Biotin | | 0.20 | 0.0073 |
| D-Ca pantothenate | 4.00 | 0.25 | 0.50 |
| Choline Chloride | 4.00 | 3.00 | 14.00 |
| Folic Acid | 4.00 | 1.00 | 1.30 |
| I-Inositol | 7.20 | 35.00 | 18.00 |
| Niacinamide | 4.00 | 1.00 | 0.036 |
| Para-aminobenzoic Acid | | 1.00 | |
| Pyridoxine HCl | | 1.00 | 0.06 |
| Pyridoxal HCl | 4.00 | | |
| Riboflavin | 0.40 | 0.20 | 0.037 |
| Thiamine HCl | 4.00 | 1.00 | |
| Vitamin B₁₂ | | 0.005 | 1.40 |

In yet another aspect, the invention provides therapeutic methods utilizing SkMCs, including (but not limited to) methods of treating myocardial infarction by transplantation of autologous or allogeneic SkMCs (e.g., in human) propagated according to the methods of the invention. Cells propagated in TGF-β are expected to exhibit enhanced proliferation and motility during the initial stages of engraftment and result in improved cardiac function.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The examples do not in any way limit the invention.

### EXAMPLES

### Example 1

Derivation of HuSkMCs Strains

HuSkMCs were derived from quadriceps muscle of a 25 year old male cadaver (Strain A), rectus femoris muscle of a 77 year old female amputee (Strain B), quadricep muscle of a 36 year old female cadaver (Strain C), or vastus laterus muscle of a 45 year old male cadaver (Strain D). Cadaver tissue, provided by the National Disease Research Institute (NDRI, Philadelphia, Pa.), was procured 8 to 19 hours post-mortem. Skeletal muscle was shipped and maintained at 0-4° C. for 2-4 days in University of Wisconsin's Solution or Iscove's Modified Dulbecco's Medium (IMDM). Then muscle was trimmed of obvious connective tissue and fat and rinsed in phosphate buffered saline (PBS). The trimmed muscle, with a wet weight of at least 4 grams, was minced into pieces of approximately 1 mm³. The minced muscle was digested in type II Collagenase (Worthington, Lakewood, N.J.) at 470 U/ml, using 15-30 ml digestion solution per gram muscle, at 37° C. for 1 hour with intermittent agitation. Cells and incompletely digested tissue were collected by centrifugation at 450 g for 7 minutes and the pellet was digested with 0.25% trypsin, 1 mM EDTA (Invitrogen, Carlsbad, Calif.) at 37° C. for 20 minutes. Digestion was stopped with fetal bovine serum (FBS) and the cell suspension was filtered through a 100 μm filter to remove incompletely digested tissue. The cell filtrate was pelleted and resuspended into culture medium (see Example 2). The yield from each 9-11 mg of trimmed muscle was inoculated per 1 cm² of BioCoat™ Collagen-I coated tissue culture flasks (Becton Dickinson, Franklin Lakes, N.J.) for propagation in 1st passage. In some cases, a one-hour pre-plating step was used, which reportedly enriches for myoblasts by taking advantage of the more rapid attachment of fibroblasts. One day later, culture medium-with unattached cells and tissue particles was replaced with fresh medium.

### Example 2

Propagation of HuSkMCs

All cultures were propagated in a 37° C., 5% CO₂, humidified environment, using collagen-I coated flasks. Medium for propagation was composed of Ham's F-12 containing GLUTAMAX™ (Invitrogen, Carlsbad, Calif.), 50 μg/ml gentamicin, 1 μg/ml amphotericin B, 15-20% FBS (Cat. No. SH30071; Hyclone, Logan, Utah), and basic fibroblast growth factor (bFGF; R&D Systems, Minneapolis, Minn.). The bFGF concentration was 5 ng/ml, except that 20 ng/ml bFGF was used for the entire propagation of Strain D and for Strain A propagation after 1st passage. The inoculation density after 1st passage was 5×10³ cells/cm². TGF-β2 (Genzyme, Cambridge, Mass.) was added as indicated in other Examples. Cultures received fresh medium every 2-4 days. When 70-100% confluent, at a density ranging from 8×10⁴ to 1.5×10⁵ cells/cm², cells were detached with 0.05% trypsin, 0.5 mM EDTA and the cell suspensions were subcultured, or analyzed as described below. In some cases, cells were cryopreserved between passages in 10% dimethylsulfoxide, 40% FBS, 50% culture medium. Studies were performed in 2nd or 3rd passage. The duration of each passage ranged from 4 to 7 days.

In a separate study, the amounts of active TGF-β1 and -β2 in one lot of 10% FBS, were quantified using ELISA-based Quantikine™ kit (Catalog No. DB100and DB250, R&D Systems, Minneapolis, Minn.). The active form of TGF-β1 and TGF-β2 were below the detection level of less than 31 pg/ml (0.031 ng/ml), while the amounts of total TGF-β1 and TGF-β2, measured after acidification of TGF-β, were 1.1 ng/ml and 0.18 ng/ml, respectively.

### Example 3

Immunolabeling Procedures for Flow Cytometry

Indirect fluorescent immunolabeling was performed to detect desmin or TE7. HuSkMCs suspensions were fixed with 4% paraformaldehyde in PBS for 20 minutes at 20-25° C. Fixed cells were washed and incubated 30 minutes at 20-25° C. with mouse anti-desmin antibody (clone D33; Dako Corp, Carpenteria, Calif.) at 2.5-5.0 μg/ml in 0.1% saponin, 10% FBS in PBS (saponin permeabilization buffer (SPB)) or with mouse “anti-fibroblast” antibody (clone TE7; Research Diagnostics, Flanders, N.J.) at 2.2 to 4.0 μg/ml in SPB. Cells were then washed and incubated 30 minutes at 4° C. with fluorescein isothiocyanate (FITC)-conjugated goat anti-mouse-IgG antibody (Jackson Immunoresearch, West Grove, Pa.) at 14 μg/ml in SPB.

Direct fluorescent immunolabeling was performed to detect CD56. HuSkMCs suspensions were incubated 30 minutes at 4° C. with phycoerythrin (PE)-conjugated mouse anti-CD56 antibody (clone NCAM16.2, BD BioSciences, San Jose, Calif.) at 1.25 μg/ml in PBS.

Dual fluorescent immunolabeling was performed to detect co-expression of desmin and CD56. After labeling HuSkMCs with PE-conjugated anti-CD56 antibody, the cells were fixed with paraformaldehyde as above and washed in PBS. Then, the fixed cells were incubated 30 minutes at 4° C. with FITC-conjugated mouse anti-desmin antibody (clone D33, Dako Corp, Carpenteria, Calif.) at 2.5 μg/ml in SPB.

All incubations were performed on cell suspensions with continuous rocking. PBS was used for all washes and immunolabeled cells were stored in PBS at 4° C. for flow cytometry.

### Example 4

Flow Cytometry

Cells were analyzed using a FACStar Plus™ flow cytometer (Becton Dickenson, San Jose, Calif.). Data acquisition of 10,000 events per sample was done without gating. Data was analyzed using CellQues™ software (Becton Dickinson, San Jose, Calif.). HuSkMCs immunolabeled with an isotype-matched negative control antibody were analyzed as a reference for CD56. A cryopreserved cell bank of a HuSkMCs strain was prepared as a reference standard for desmin and TE7 immunolabeling and flow cytometric analysis. A cell sample from the reference bank was thawed, immunolabeled, and analyzed by flow cytometry for each study reported. The reference standard, tested in 21 independent assays, was on average 52.7% desmin-positive (coefficient of variation=6.2%) and 46.1% TE7-positive (coefficient of variation=6.2%).

In density plots of fluorescence versus forward scatter, the positive population was quantified within a polygonal region bounded on one side by the straight line that best separated the negative and positive populations. In histograms, the positive population was quantified by setting a region marker beginning at the nadir between the negative and positive peaks and extending to the upper end of the fluorescence intensity scale.

### Example 5

Visualization of Myotubes

HuSkMCs were propagated as above, except cells were inoculated into slideflasks without collagen-coating (Nunc, Denmark). When the culture was confluent, it was maintained for two weeks in 1% FBS with basal medium and antibiotics described above. The attached cell monolayer was then fixed and subjected to indirect fluorescent immunolabeling for detection of desmin as described above for cell suspensions except incubation periods were increased 50% and more extensive rinsing with PBS was performed between incubations. The microscope slide of the slideflask was detached and coverslipped using a mounting medium containing 4′, 6-diamidino-2-phenylindole (DAPI; Vector Labs, Burlingame, Calif.). Mounted cells were photographed under 100× magnification using a fluorescent microscope, and images of FITC (desmin) and DAPI (nuclei) were overlaid.

### Example 6

Creatine Kinase Assays

Assays were performed on HuSkMCs propagated in serum-rich media (described above) or after differentiation. Differentiation was induced by seeding at a density of 8×10⁴ cells/cm² into standard tissue culture flasks and culturing in propagation medium for 1 day, then in 2% FBS for the period indicated.

Pellets of approximately 2×10⁶ cells were lysed by suspension in 75 μl 0.2% Triton X-100™ in PBS (pH 8.0) for 10 minutes at 20-25° C. Sub-cellular particles were removed by centrifugation at 16,000 g for 20 minutes at 4° C. and the supernatant mixed 1:1 with 20 mM glycine in PBS, pH 8.0. Samples were aliquoted and stored at −80° C. for quantification of creatine kinase activity and total protein.

A reagent mixture for determination of creatine kinase activity was used in a kinetic assay according to manufacturer's instructions (Procedure # 47-UV, Sigma, St. Louis, Mo.). By this method, creatine kinase in the cell extracts was combined with the reagent mixture of substrates and enzymes to initiate a series of enzymatic reactions that ultimately produced NADH, which increased absorbance at 340 nm. Data were accepted for consideration only when the correlation coefficient for abs₃₄₀/time was greater than 0.99. Each cell extract was tested in triplicate wells of a 96-well microtiter plate. Creatine kinase activity was normalized to total protein, which was measured against a bovine serum albumin standard curve in a Bradford assay. Absorbance readings for both assays were performed directly in microtiter wells using a Spectramax™ Plus³⁸⁴ spectrophotometer (Molecular Devices, Sunnyvale, Calif.).

A reference standard for the above assays, an extract from a differentiated HuSkMC culture was prepared as above, aliquoted, and stored at −80° C. The reference standard was tested in 46 independent assays over a period of more than 4 months. The assay results for the reference standard, which was included with all creatine kinase assays, averaged 0.724 creatine kinase units/mg protein, with a coefficient of variation of 7.8% and showed no loss of activity in storage.

### Example 7

Northern Analysis

HuSkMC suspensions were pelleted, snap frozen in RNAlater™ (Ambion, Austin, Tex.), and stored at −80° C. RNA was isolated using the protocols included in the QiaShredder™ (Qiagen, Valencia, Calif.) and RNeasy™ (Qiagen, Valencia, Calif.) kits, and quantified by measuring absorbance at 280 nm. RNA was resolved by electrophoresis in a 1% agarose, 5% formaldehyde gel, after loading 8 μg per well. The RNA was transferred from the gel to a nylon membrane, and probed with a ³²P-labeled 780-nucleotide fragment of human desmin cDNA. Desmin mRNA was quantified using a BAS-1500 phosphoimager (Fugifilm, Stanford, Conn.) and ImageGuage™ V3.46 software (Fugifilm).

### Example 8

HuSkMC Cultures are Mixed Populations of Myoblasts and Fibroblasts

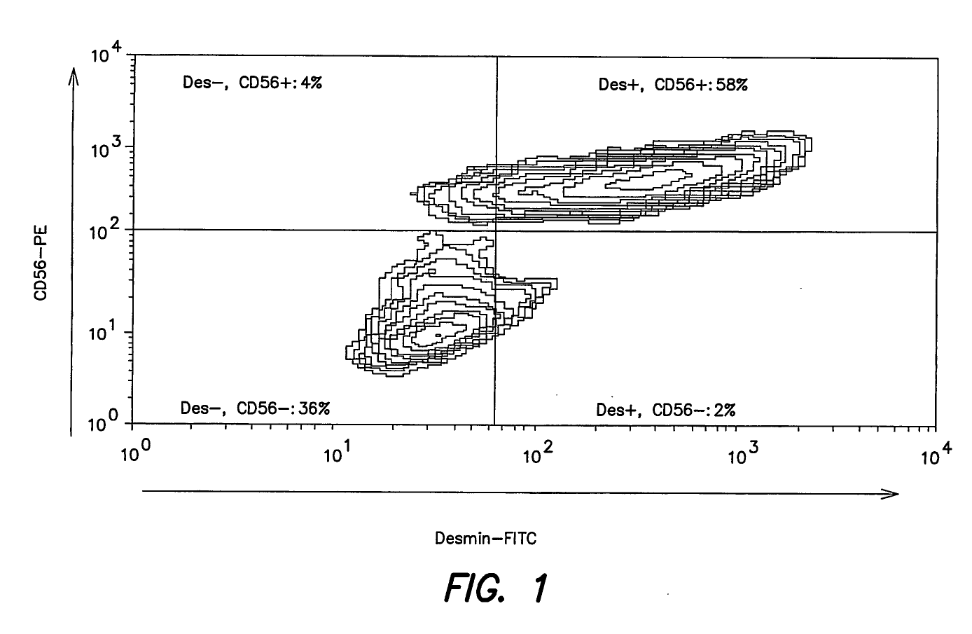

HuSkMCs were cultured in collagen-coated flasks as described for propagation of HuSkMCs. On third passage, dual fluorescent immunolabeling for the myoblast markers desmin and CD56 (Kaufman et al. (1988) Proc. Natl. Acad. Sci. USA, 85:9606-9610; and Belles-Isles et al. (1993) Eur. J. Histochem., 37:375-380) was performed. HuSkMC cultures from more than 20 donors were analyzed by flow cytometry. The results revealed that cultures were typically composed of two major populations of cells: one expressing both desmin and CD56 markers (i.e., myoblasts) and the other expressing neither marker. Results of flow cytometric analysis for a representative culture (strain A) are shown in FIG. 1.

To confirm the presence of differentiation-competent myoblasts in the propagated HuSkMC cultures, cells were subjected to conditions that enhance myoblast differentiation, i.e., culture in low-serum. Specifically, HuSkMCs were cultured in 1st passage in collagen-coated flasks as described for propagation of HuSkMCs. Cells were then seeded at low density onto culture flasks without collagen-coating, propagated to confluent density in 2nd passage, cells were then maintained for 2 weeks in 1% serum to promote myotube formation. The differentiated cells were fixed while attached to the culture flask. Desmin was detected by fluorescent immunolabeling, and nuclei were stained with DAPI. Multinucleate myotubes were observed indicating that myoblasts in the culture had differentiated.

To further confirm the presence of differentiation-competent myoblasts in the propagated HuSkMC cultures, 2nd passage HuSkMCs were seeded into non-coated flasks at 80,000 cells/cm², induced to differentiate in 2% serum for the duration indicated and assessed for creatine kinase activity. Creatine kinase activity increased over time indicating that myoblasts in the culture had differentiated. Results of a representative study (strain D) are shown in Table 3.

TABLE 3

| | Days | | | | |
|---|---|---|---|---|---|
| | in 2% serum | | | | |
| | 0 | 2 | 4 | 6 | 8 |
| Creatine kinase activity, | 0.22 | 0.68 | 0.96 | 1.09 | 1.44 |
| U/mg protein | | | | | |

To characterize the non-myoblast population of HuSkMCs, HuSkMCs strains of low and high myoblast purity (Stains B and C, respectively) were thawed from cryopreserved banks and were propagated through 2nd passage independently or after mixing the two strains in approximately equal proportions (Strain B+C). The 2nd passage cultures of low (Strain B), medium (Strain B+C), and high (Strain C) myoblast purity were subjected to flow cytometric analysis for quantification of cells expressing TE7 antigen or desmin. In each culture, irrespective of myoblast purity, the fraction of desmin-positive and TE7-positive cells totaled approximately 100%. The pattern of forward scatter by flow cytometry, a measure of cell size, was similar between the desmin-negative and TE7-positive populations. Taken together, the data indicate that the expression of desmin and TE7 antigen was mutually exclusive. No endothelial or fat cells in HuSkMC cultures were detected using the acetylated-LDL uptake assay (Voyta et al. (1984) J. Cell Biol., 99(6):2034-2040) and Oil Red O assay (Kuri-Harcuch et al. (1978) Proc. Nati. Acad. Sci. USA, 75(12):6107-6109) with the appropriate cells as positive controls. The data indicates that propagated HuSkMCs were comprised almost entirely of two major cell populations, namely differentiation-competent myoblasts and fibroblasts.

### Example 9

Effects of TGF-β During Propagation of HuSkMCs

To determine the effects of TGF-β on cell growth and differentiation of HuSkMCs, cells of strain A were propagated in 2nd passage and exposed to 1 ng/ml TGF-β2 for different intervals, each extending to the termination of a 5 day culture period. Cells were then immunolabeled and analyzed by flow cytometry for quantitative detection of desmin and CD56 expression as described above. While the pattern of fluorescence intensity of the desmin-negative peak was unaffected by TGF-β2, the fluorescence intensity of the desmin-positive peak showed a progressive decline as the time of exposure to TGF-β2 increased. This change reflected a decrease in desmin expression in the myoblast population.

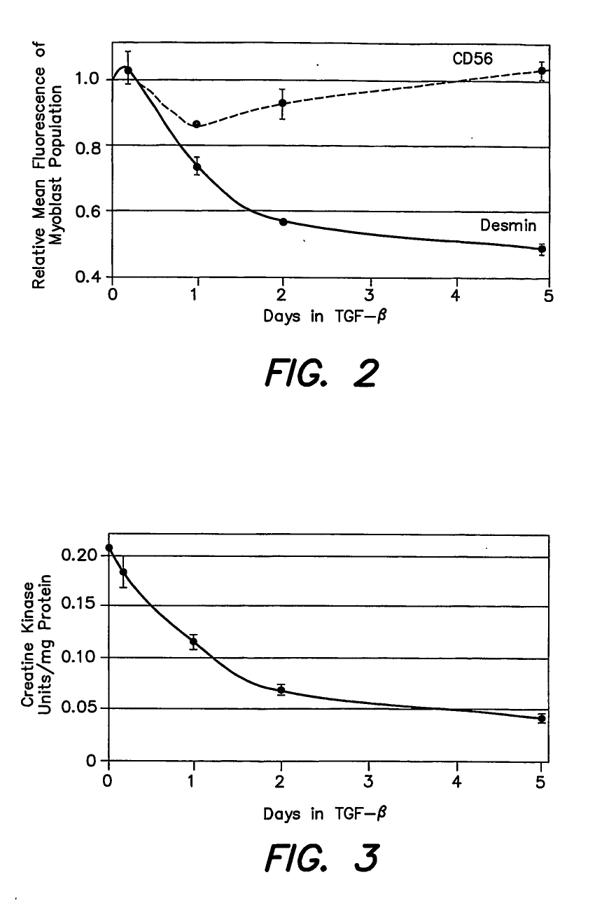

Quantification of the flow cytometry results (FIG. 2) showed that the average fluorescence intensity of the myoblast population of HuSkMCs exposed to TGF-β2 for five days was 48% of that for untreated cells. Approximately half the decrease in desmin expression occurred after one day of exposure to TGF-β2. The observed decrease in desmin expression in response to TGF-β2 was further supported by results from Northern analysis of the cells from the same strain propagated 4 days in duplicate either in the presence or absence of 1 ng/ml TGF-β2. Northern blots for detection of desmin mRNA were prepared and quantified as described above. The average intensity of signal from the bands of the Northern blot corresponding to desmin RNA from cultures exposed to TGF-β2 was 53% of the average signal from cultures propagated in the absence of TGF-β2 (146 and 194 pixels versus 310 and 327 pixels, respectively).

In contrast, TGF-β2 treatment did not alter the fluorescence intensity of the CD56-positive population, indicating that desmin and CD56 are regulated independently of each other. Furthermore, the fraction of the culture represented by CD56-positive cells was similar between HuSkMCs propagated in the absence and presence of TGF-β2 (65% and 63%, respectively). In a separate study, expression of the fibroblast marker TE7 was also unaffected by TGF-β2. The data suggests that TGF-β2 does not alter the ratio of the total number of fibroblasts and myoblasts within the culture.

### Example 10

Reversibility of TGF-β-Induced Downregulation of Desmin

To determine whether TGF-β2-induced the decline in desmin expression was reversible, HuSkMCs of Strain C were propagated 5 days in 2nd passage in the absence or presence of 1 ng/ml TGF-β2 medium, then harvested for fluorescent immunolabeling and flow cytometric analysis for the detection of desmin. Parallel cultures were propagated in TGF-β2, then cultured in the absence of TGF-β2 for 2 additional days before harvesting. The results are summarized in Table 4.

TABLE 4

| | Culture Conditions | | |
|---|---|---|---|
| | | | +TGF-β2 followed |
| | no TGF-β | +TGF-β2 | by no TGF-β |
| Relative mean fluorescence | 1.0 | 0.51 | 0.95 |
| of myoblast population | | | |
| Desmin+ cells | 88 | 82 | 83 |
| (percent of total) | | | |

As shown in Table 4, the mean fluorescence of the desmin-positive population from the TGF-β2-treated cultures was about 50% of that from the untreated cultures. However, 2 days after removal of TGF-β2, the culture acquired a profile of desmin expression similar to that of cells never exposed to TGF-β2. The fraction of cells with a fluorescence intensity corresponding to the desmin-positive population was similar among the 3 cultures. The data indicates that continuous exposure to TGF-β2 is required for suppression of the myoblast marker desmin and that the normal myoblast phenotype can be reestablished within 2 days by removal of the TGF-β2.

### Example 11

Effect of TGF-β on Creatine Kinase Activity

The modulation of desmin by addition and removal of TGF-β2, indicates that TGF-β can be used to control the state of differentiation of myoblasts during propagation of HuSkMCs. To assess this further, the effect of TGF-β2 on creatine kinase activity was investigated. Creatine kinase levels were quantified directly from samples taken from the same strain A cultures used to examine the down-regulation of desmin by flow cytometric analysis shown in FIG. 3. TGF-β2 reduced creatine kinase activity at a rate similar to that observed for desmin, with approximately half of the reduction occurring after 1 day of TGF-β2 treatment (compare FIG. 3 with FIG. 2).

In a separate study, the reversibility of TGF-β2-induced down-regulation of creatine kinase was assessed. HuSkMCs of strain A were propagated 5 days in the absence (culture 1) or presence (cultures 2, 3, and 4) of 1 ng/ml TGF-β2. One of the TGF-β2-treated cultures was propagated an additional 2 days in TGF-β2 (culture 3) and one was cultured an additional 2 days in its absence (culture 4). At the end of each culture period, cells were lysed for creatine kinase analysis. HuSkMCs of strain A cultured 5 days in the presence of TGF-β2 (Table 5, culture 2) had a creatine kinase activity that was 15% of the activity in cells cultured in its absence (Table 5, culture 1). When these cells were propagated an additional 2 days without TGF-β2 (Table 5, culture 4), the creatine kinase activity increased 15-fold after TGF-β2 removal (compare cultures 2 and 4), demonstrating that TGF-β2 did not permanently block the expression of this muscle differentiation marker. Since myoblasts tend to differentiate when confluent, the large increase in activity following the removal of TGF-β2 in culture 4 may be partly due to the high cell density, 2.1×10⁵ cells/cm², achieved at the end of the culture period. However, when TGF-β2-treated cells were cultured an additional 2 days in the presence of TGF-β2 for a total of 7 days continuous exposure to the growth factor (Table 5, culture 3), creatine kinase activity remained low, even though these cells also attained a high density (2.3×10⁵ cells/cm²), similar to that of culture 4. This data, combined with the data from flow cytometric analysis of desmin expression, indicates that TGF-β2 suppresses myoblast differentiation, even in high density HuSkMC cultures. Moreover, the combined data shows that this effect of TGF-β2 is fully reversible and suggests that HuSkMCs propagated in TGF-β2 retain their capacity to differentiate.

TABLE 5

Culture

| | 1 | 2 | 3 | 4 |
|---|---|---|---|---|
| Creatine kinase activity | 0.28 | 0.04 | 0.07 | 0.59 |
| U/mg protein | | | | |

### Example 12

Transplantation of Skeletal Muscle Cells into Infarcted Myocardium

This study compares the clinical effect of transplanted skeletal muscle cells (SkMCs) after in vitro propagation in the presence or absence of TGF-β in a non-human animal (e.g., Lewis rats) intended as a model of post-infarction heart function in human. The cells used in this study are cultivated and stored as cryopreserved cell banks prior to transplantation. Optionally, two to three days prior to harvest of skeletal muscles as a source of SkMCs, 0.5 ml Marcaine™ (0.5% bupivicaine chlorohydrate) can be injected into the anterior tibialis of each hindleg of anesthetized rats. This procedure activates satellite cells and thereby enhances baseline myoblast cell yield from subsequent in vitro cultures.

Studies to assess the survival of donor cells transplanted into syngeneic recipients can be optionally conducted in a pilot study. Briefly, SkMCs are labeled using fluorescent vital dyes. Two groups of non-infarcted rats are transplanted with the labeled cells and after 1 week, the animals are sacrificed and their hearts paraformaldehyde fixed and analyzed through histology for SkMC cell survival or evidence of inflammatory infiltrates. Fluorescent labeling of cells is performed as follows. After thawing a frozen cell ampule and dilution with 3 ml 80% IMDM, 20% FBS the cells are concentrated by centrifugation at 160-200 g for 5 minutes, as described above. The cell pellet is suspended in 10 ml of labeling medium consisting of 1 μM dioctadecyloxacarbocyanine perchlorate (DiO) (Molecular Probes; Eugene, Oreg.) prepared in HBSS (Ca⁺/Mg³⁰ -free). The 10 ml cell suspension is incubated for 5 minutes at 37° C., in the dark, followed by a 15 minute incubation at 4° C.

In the main study, the day before surgery (day-1) rats are assigned to one of two groups: sham or infarction. Sham animals are evaluated for cardiac function with 2D-guided M-mode echocardiography. On the day of surgery (day 0) animals are anesthetized and hearts exposed via anterolateral thoracotomy. A suture ligature is secured around the LAD and tightened to create an ischemic injury only in animals assigned to the infarct group. Animals assigned to the sham group will complete the thoracotomy but will not be infarcted thus serving as a control group. The infarction group is then subjected to profound myocardial ischemia (infarction) by coronary artery ligation for 60 minutes using a suture followed by re-perfusion. Six days after infarction, all animals are weighed and assessed for exercise tolerance on a treadmill. Seven days after infarction, all animals are evaluated for ejection fraction using echocardiography. Eight days after infarction all animals are operated on again, in the same order as the initial surgery, to re-expose the heart. Infarcted animals are assigned to one of three subgroups according to the transplant they receive: (1) placebo injection of cell suspension medium without cells); (2) SkMCs cultivated in the presence TGF-β(e.g., TGF-β1, -β2, and/or -β33) as per methods of the invention; and (3) SkMCs cultivated without TGF-β. The sham group is subjected to the second thoracotomy but does not receive any injection. Each rat in the SkMCs group receives 6-10 injections (total of 3×10⁶ cells/heart) of cell suspension, contained in a total volume of 100 μl of IMDM/0.5% BSA, directly into the infarct and peri-infarct region approximately 1-2 mm apart, using a 30 gauge Hamilton needle.

Following treatment, the thorax is closed and the animal allowed to recover. Animals are examined daily and signs of cardiac failure (lethargy, shallow breathing, cyanosis) and mortality, noted. The weight of each animal is recorded weekly and immediately prior to any analytical procedure. Death of any animal during the study is recorded and subjected to necropsy to determine likely cause of death.

Eight weeks after transplantation the animals are assessed for exercise capacity using a treadmill. Maximum exercise capacity is measured as the distance run on a modified rodent treadmill (Columbus Instruments; Columbus, Ohio) until exhaustion. Exhaustion is defined as the inability to run for 15 consecutive seconds despite minor electric shock. Initial treadmill speed is set at 15 meters/minute at a 15° grade and increased by 1 meter/min increments every minute thereafter.

Cardiac function is examined with 2D guided M-mode echocardiography to determine left ventricle ejection fraction. Echocardiographic assessment of in-vivo cardiac function is conducted in anesthetized rats, using an Acuson Sequoia™ C-256 echocardiograph machine (Siemens, Malvern, Pa.) equipped with a 15 MHz probe. Animals are anesthetized through inhalation of 5% isoflorane using a rodent nose-cone, and maintained on 2.5% isoflorane throughout the echocardiogram to ensure proper anesthesia. Isoflorane allows for rapid, smooth induction of anesthesia and rapid recovery, with very little alteration of cardiovascular hemodynamics (ventricular loading, blood pressure, heart rate, etc). Once anesthetized, the animal chest is shaved using commercial electric clippers. The heart is imaged in the two-dimensional parastemal short axis view and an M-mode measurement recorded at the mid ventricle at the level of the myocardial infarct. The heart rate, anterior/posterior wall thickness, and the end-diastolic/end-systolic cavity dimensions are measured from the M-mode image using commercially available analysis software (Acuson Sequoia). Fractional shortening is defined as the end-diastolic dimension minus the end-systolic dimension normalized for the end-diastolic dimension, and is used as an index-of cardiac contractile function. Regional anterior and posterior wall thickening are also assessed through comparison of diastolic and systolic wall dimensions of the respective regions. Parameters of diastolic function and ventricular filling, including early/late LV blood inflow (E/A ratio) and rate of blood inflow, are measured through Doppler measurements of blood velocity across the mitral valve. (Cardiac function in regional myocardial segments in larger animals can be assessed using magnetic resonance imaging (MRI).)

The animals will then be anesthetized and their hearts excised followed by cardiac performance analysis of developed pressure using a Langendorff perfusion system on cultured isovolumically beating (balloon-in-LV) hearts. Briefly, cultured hearts are retrogradely perfused with a perfusate consisting of bovine red-blood cells suspended in modified Krebs-Henseleit buffer at a hematocrit of 40%. A fluid-filled cling-film balloon connected to a Statham P23Db™ pressure transducer (Statham Instrument, Hato Rey, Puerto Rico) is placed into the left ventricle to monitor ventricular pressures. Coronary perfusion pressure is set to 80 mm Hg and active pressure-volume relationships then generated. From a balloon volume of zero, the balloon is filled in increments of 0.05 ml and subsequent peak systolic and end-diastolic pressures are recorded. Systolic and diastolic pressure-volume relationships will then be derived. Subsequently, the hearts are arrested in the diastolic state and at a final distending pressure of 5 mm Hg with potassium chloride, and fixed by retrograde perfusion with 4% paraformaldehyde.

Following fixation, the hearts are trimmed of atrial tissue, weighed, and tranversly cut (“bread-loaved”) into four equal segments. The heart segments are embedded in paraffin and cut into.5 μm thin sections for Masson's trichrome histochemistry and scar area determination by planimetry. Skeletal muscle tissue is identified on the basis of skeletal myoblasts present in the transplant mixture that are anticipated to differentiate into skeletal myofiber cells. The identification of skeletal muscle cells is performed immunohistochemically using a skeletal muscle-reactive anti-myosin heavy chain antibody that does not stain cardiac muscle (for example, MY-32 antibody (Sigma-Aldrich, St. Louis, Mo.) described in Havenith et al. (1990) Histochemistry 93:497-499).

It is predicted that cardiac function in rats treated with SkMCs cultured in TGF-β is equal or better (by at least 10, 20, 30, 40, 50, 60, 70, 80, 90, 100, 150, 200, 300, 500% or more) relative to the control group(s) and/or as compared to similar cells cultured without TGF-β. Additionally, it is predicted that cells propagated in TGF-β exhibit enhanced proliferation and motility during the initial stages of engraftment.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents and sequences cited in this disclosure are incorporated by reference in their entirety. To the extent the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A method of propagating adult mammalian skeletal muscle cells, the method comprising culturing the cells in a mitogen-rich cell culture medium supplemented with an amount of TGF-β effective to reversibly suppress myoblast differentiation.

2. The method of claim 1, wherein the skeletal muscle cells are human.

3. The method of claim 1, wherein the cell culture medium comprises at least 5% serum.

4. The method of claim 1, wherein TGF-β is one of, or any combination of, TGF-β1, TGF-β2, and TGF-β3, or heterodimers thereof.

5. The method of claim 1, wherein the effective amount of TGF-β is from 0.01 to 200 ng/ml.

6. The method of claim 1, wherein the skeletal muscle cells are primary cells.

7. The method of claim 1, wherein the skeletal muscle cells are passaged.

8. The method of claim 1, wherein the skeletal muscle cells are cultured in the presence of TGF-β for at least 12 hours.

9. The method of claim 1, wherein the skeletal muscle cells are grown to over 30% confluence prior to passaging or harvest.

10. The method of claim 1, wherein the skeletal muscle cells are grown to cell density of over 0.1×10⁵ cells/cm².

11. The method of claim 1, wherein expression of creatine kinase by skeletal muscle cells is reduced by at least 20% relative to a control culture propagated without the supplementation with TGF-β.

12. The method of claim 1, wherein expression of desmin by CD56-positive myoblasts is reduced by at least 20% relative to CD56-positive myoblasts propagated without the supplementation with TGF-β.

13. The method of claim 1, wherein expression of creatine kinase by skeletal muscle cells is reduced by at least 20% relative to the same culture of skeletal muscle cells prior to the addition of TGF-β.

14. The method of claim 1, wherein expression of desmin by CD56-positive myoblasts is reduced by at least 20% relative to CD56-positive myoblasts in the same culture of skeletal muscle cells prior to the addition of TGF-β.

15. Cells produced by the method of claim 1.

16. A method of treating myocardial infarction, comprising transplanting the cells of claim 15 into infarcted myocardium.

17. The method of claim 16, wherein the cells are autologous or allogeneic.

18. Cultured skeletal muscle cells expressing normal levels of CD56 and reduced levels of desmin, wherein desmin expression is at least 20% lower than in the primary culture.

19. Cultured skeletal muscle cells expressing normal levels of CD56 and reduced levels of desmin, wherein desmin expression is at least 20% lower than in a control culture propagated without TGF-β.

20. Cultured skeletal muscle cells expressing normal levels of CD56 and reduced levels of expression of desmin, wherein desmin expression is at least 20% lower than that in the culture prior to the addition of TGF-β.

21. A method of treating myocardial infarction, comprising transplanting the cells of claim 18 into infarcted myocardium.

22. The method of claim 16, wherein the cells are autologous or allogeneic.

23. A method for evaluating the differentiation state of myoblasts in a skeletal muscle cell culture, the method comprising determining the amount of desmin expressed by a population of CD56-positive cells in the skeletal muscle cell culture, wherein the amount of desmin below a threshold level indicates the presence of undifferentiated myoblasts in the SkMC culture.

24. The method of claim 23, wherein the amount of desmin is determined using fluorescence-activated cell sorting.
